(19) United States
(12) Patent Application Publication
(10) Pub. No.: US 2022/0401508 A1
(43) Pub. Date: Dec. 22, 2022

(54) COMPOSITION COMPRISING SWEET POTATO STEM OR LEAF EXTRACT AS ACTIVE INGREDIENT FOR PREVENTION OR TREATMENT OF EAR DISEASE

(71) Applicant: Vivoin Co., Ltd., Gyeonggi-do (KR)

(72) Inventors: Ha Na Hong, Gyeonggi-do (KR); Woo Jae Jung, Seoul (KR)

(21) Appl. No.: 17/771,438

(22) PCT Filed: Sep. 28, 2020
(86) PCT No.: PCT/KR2020/013185

§ 371 Date: Apr. 22, 2022

(30) Foreign Application Priority Data
Oct. 24, 2019 (KR) 10-2019-0132777

(51) Int. Cl. A61K 36/81; A61K 31/7036; A61K 31/045; A61P 27/16

(52) U.S. Cl. CPC A61K 36/81; A61K 31/7036; A61K 31/045; A61P 27/16

(57) ABSTRACT

The present disclosure provides a composition containing a sweet potato stem and/or leaf extract for prevention, alleviation, or treatment of an ear disease. The composition according to the present disclosure can suppress hearing loss by effectively inhibiting the increase in hearing threshold due to hearing loss and the damage to hair cells, and is useful for the prevention or treatment of an ear disease, such as hearing loss or ringing in the ears, by having effects of reducing abnormal behavior caused by ringing in the ears and of returning the hearing amplitude to normal, and therefore, the composition can be advantageously used as a pharmaceutical composition or a health functional food.

### TECHNICAL FIELD

The present disclosure relates to a composition comprising a sweet potato stem or leaf extract for prevention or treatment of an ear disease.

### BACKGROUND ART

Most of the research and development of ear-related diseases were directed to diseases caused by inflammation, such as bacterial otitis externa, otitis malignant, fungal otitis externa, otomycosis, otitis media, otitis interna, and the like. Thus, technological development of related therapeutic agents has been mainly carried out.

Specifically, otitis externa (outer ear infection) is a condition that causes inflammation of the outer ear and the ear canal. The otitis externa is a common cause of earache in humans, and the inflammation may be caused by fungal, viral or bacterial organisms. The skin of the ear canal often swells, is painful, and may hurt to the touch. Otitis media (middle ear infection) occurs in the area between the eardrum and the inner ear including the Eustachian tube. The otitis media is very common in children. On average, infants suffer from otitis media 2-3 times a year, usually accompanied by viral upper respiratory infections (URIs), and most often by colds. Rhinoviruses and adenoviruses, which are responsible for the very common cold symptoms, cause swelling and redness in the inner ear. The otitis media is also commonly caused by various bacteria and other viruses.

In most of the treatment of ear-related diseases as above, antibiotics, anti-inflammatory drugs, and the like, were used as drugs. Even today, most therapeutic agents are based on treatment methods standing on the basis of antibiotics or anti-inflammatory drugs.

However, in modern society, due to the increase in exposure to noise, the aging of the population, and the like, problems related to hearing loss caused by damage to cells or nerves are on the rise rather than the above inflammation-based ear-related diseases. Moreover, even most ordinary people are able to distinguish the difference between ear-related diseases that are caused by inflammation, for example, bacterial otitis externa, otitis malignant, fungal otitis externa, otomycosis, otitis media, and otitis interna, and hearing loss diseases caused by damage to cells or nerves. Hearing loss is one of the most common diseases with a high incidence in modern society. The hearing loss due to cell or nerve damage is caused by ototoxic drugs, neurotoxic chemicals, viruses, genetics, and the like, in addition to noise, aging, and the like, and sometimes the cause thereof is also unknown.

For the treatment of hearing loss as described above, a number of specialists and pharmaceutical industry-related practitioners still aim to treat the above-described diseases with therapeutic agents based on existing antibiotics or anti-inflammatory drugs, but these therapeutic agents may not be suitably used for hearing loss. The first reason for using the existing therapeutic agents is that there are no known therapeutic agents for hearing loss, and the second one is that many specialists and pharmaceutical industry-related practitioners are not clearly aware of treatment methods for hearing loss. Due to the above-described problems, failure to properly perform treatment for hearing loss continues to occur.

In particular, the use of antibiotics causes ototoxicity, which results in great problems. For example, aminoglycoside antibiotics have ototoxicity and nephrotoxicity as side effects that cause hearing and balance dysfunction in the inner ear, which may occur not only in an overdose, but also in long-term use at a therapeutic dose, and in some cases, the ototoxicity may occur even at an appropriate dose for a short period of time. The ototoxicity of aminoglycoside antibiotics shows vestibular dysfunction in about 15% of users and hearing loss in 10-30% of users, and mainly occurs in both ears in the form of sudden severe hearing loss at high frequencies above 4000 Hz.

Accordingly, preclinical studies on various drugs have been reported as studies to find substances effective for the prevention and treatment of hearing loss. However, it has been confirmed that there are limitations to proceed with clinical trials. In addition, there are no drugs approved for the prevention and treatment of hearing loss to date. Moreover, there are almost no drugs developed for therapeutic agents of hearing loss using natural products or natural materials capable of being used without toxicity or risk to the human body. In other words, unlike ear-related diseases caused by inflammation, a suitable therapeutic agent for hearing loss has not been developed at all.

On the other hand, tinnitus is often referred to as ringing in the ears (perception of sound in the absence of an external source of an acoustic signal). Tinnitus, syrigmus or ringing in the ears is the perception of sound in the human ear in the absence of a corresponding external sound. For a simple explanation, tinnitus, syrigmus or ringing in the ears is a sound that is heard from the inside even though it is not coming from outside (Jastreboff et al., J Am Acad Audiol,. 11(3), pp162-177, 2000).

This tinnitus may appear briefly and then disappear, may persist, or may be perceived as a continuous sound. In the tinnitus, the pitch of the sound also varies, and it is usually found in one ear or may also occur in both ears. Statistically, about 15 to 20% of adults experience various types of tinnitus, of which 4% have experienced severe tinnitus. In addition, it is also known that 70-80% of people with hearing loss experience tinnitus (Demeester K et al., B-ENT., 17, 37-49, 2007).

Tinnitus may be divided into objective tinnitus (or non-subjective tinnitus) that is audible even from the outside and subjective tinnitus (or conscious tinnitus) that is audible to the patient himself but is not audible from the outside. In addition, tinnitus may be classified into peripheral tinnitus and central tinnitus based on differences in how they are perceived by the affected individual. The peripheral (or cochlear) tinnitus is presumed to originate from the peripheral nervous system and cochlea, and the central tinnitus is presumed to originate from the auditory cortex.

The objective tinnitus, which allows doctors to hear an actual sound coming from the patient's ear, may produce a ‘click’ or ‘tack’ sound from the muscles around the middle ear due to spasm. Some people even hear the sound according to the pulse. Pulsatile tinnitus may result from changes in blood flow or the flow of veins around the ear (Chandler JR, Laryngoscope, 93, pp892-895, 1983), however, it can be a subjective symptom caused by increased interest in blood flow in the ear. To date, the pathophysiological characteristics of subjective tinnitus are poorly understood, and the exact pathogenesis thereof is unknown.

Since tinnitus is usually a subjective phenomenon, it is difficult to measure tinnitus with the otologic hearing evaluation method for general evaluation of tinnitus. This difficulty was regarded as a factor that did not succeed in developing a therapeutic agent for tinnitus as it acted as a limiting point in animal experiments for tinnitus. In clinical practice, the evaluation of tinnitus is conducted by scoring the patient's subjective pain through the questionnaire. The condition is often clinically rated on a simple metric scale from ‘mild’ to ‘severe’ depending on practical difficulties such as sleep, meditation, interruption of daily activities, and the like. Persistent tinnitus may lead to irritability, fatigue, and sometimes clinical depression, and may also cause auditory hallucinations (McCombe et al., Clin Otolaryngol., 26(5), pp388-393, 2001.; Davis et al., Epidemiology of Tinnitus. In: Tyler R, editor. Tinnitus Handbook. San Diego: Singular Publishing Group; 2000. pp1-23).

In clinical practice, there is no objective evaluation tool for tinnitus, and tinnitus is evaluated depending on the subjective evaluation method complained of by the patient. Most patients with tinnitus are diagnosed with subjective tinnitus, and treatment for subjective tinnitus has been focused on relieving tinnitus through rehabilitation so far. For example, there are a tinnitus masking method in which a sound similar to the patient's tinnitus is heard at a louder volume than the tinnitus from the outside through a hearing aid-like device, so that the tinnitus is not heard, a tinnitus retraining therapy that treats tinnitus without accompanying hearing loss by continuously playing sounds smaller than the actual tinnitus over a wide frequency range, and the like. To date, there is no treatment for tinnitus approved by the US Food and Drug Administration (FDA).

Under this background, there is an urgent need for research and development on a composition for preventing or treating an ear disease using a material capable of being easily used as a material for food or pharmaceuticals.

Sweet potato (Ipomoea batatas) is a perennial herb that belongs to a dicotyledonous plant in the order Solanales and the family Convolvulaceae, and also called Ipomoea batatas (L.) Lam. It is native to South America, but the distribution area has been expanded to Asian regions such as Korea, China, and Indonesia, and thus it is one of the most important food crops grown worldwide. In the case of the sweet potato, the root of the sweet potato mainly composed of starch, i.e., the tuberous root, is used as a carbohydrate source such as food. The tuberous root of the sweet potato is rich in various vitamins, minerals and extracts in addition to starch, and is not only used for food, but also recently used as a raw material for side dish or snacks, alcoholic beverages such as industrial starch soju (Korean distilled spirits) or whiskey, alcohols, cosmetics or pharmaceuticals.

Meanwhile, as to sweet potatoes, the tuberous root is mainly used, and also mainly used for various uses, such as for preparation of alcoholic beverages, as well as for use as food, which is the main method of using sweet potatoes, and the stems or leaves of sweet potatoes are only used limitedly in Korea. In particular, since the leaves and stems wither when there is frost in the fall when sweet potato tubers are harvested, the leaves and stems are rarely harvested or treated only as by-products of sweet potato tuberous root, which are generally not used. Therefore, there is a need to develop various application methods for the stems or leaves of the sweet potato. However, unlike the research on the tuberous root of sweet potatoes among the parts of sweet potatoes, almost no research has been conducted on the stems or leaves of sweet potatoes. In particular, considering that only the tuberous root part of sweet potato is generally used and physiologically active substances of the tuberous root are very different from those of stems or leaves, there is a need for research and development on extracts from other parts of sweet potato.

Thus, as research on the treatment or prevention of an ear disease is insufficient, it is necessary to develop ingredients effective for the treatment and improvement of an ear disease in advance in order to proceed with research related to ear diseases.

Under this background, the present inventors conducted research and development on natural extracts capable of preventing, improving and treating an ear disease targeting natural materials with excellent safety, and completed the present disclosure.

### DISCLOSURE

### Technical Problem

An object of the present disclosure is to provide a pharmaceutical composition for preventing or treating an ear disease, comprising an extract of sweet potato stems, leaves, or both as an active ingredient.

Another object of the present disclosure is to provide a food composition for preventing or treating an ear disease, comprising an extract of sweet potato stems, leaves, or both.

### Technical Solution

The present inventors made great efforts to find materials capable of preventing, improving, and treating an ear disease by targeting natural materials with excellent safety, and as a result, surprisingly confirmed that sweet potato stem and/or leaf extracts effectively prevent, improve and treat ear diseases such as hearing loss and tinnitus, and completed the present disclosure.

The present disclosure provides a pharmaceutical composition for preventing or treating an ear disease, comprising an extract of sweet potato stems, leaves, or both as an active ingredient.

The pharmaceutical composition for preventing or treating an ear disease of the present disclosure has an excellent therapeutic effect on hearing loss by effectively inhibiting the increase in the hearing threshold and hair cell damage due to hearing loss.

In addition, the pharmaceutical composition for preventing or treating ear disease of the present disclosure has an excellent therapeutic effect on tinnitus by reducing abnormal behavior due to tinnitus and returning the hearing amplitude to normal.

In particular, the sweet potato stem and/or leaf extracts have a remarkable effect in the prevention and treatment of an ear disease such as hearing loss or tinnitus, and also have high economic efficiency by employing the extract of stems or leaves, which parts that have not been used before as medicinal or health functional food.

In the present disclosure, the ear disease refers to a disease that occurs in the ear, including the inner ear, middle ear, and outer ear. The ear disease includes symptoms including, but not limited to hearing loss, tinnitus, deafness, nystagmus, inflammation, edema, infection, and congestion in ear. These diseases may be caused by several causes, such as infection, injury, inflammation, tumors, and adverse reaction to drugs or other chemicals.

Preferably, the ear disease may be hearing loss, tinnitus, or both.

In the present disclosure, “hearing loss” means any state in which hearing is reduced or lost. Hearing loss may include, but is not limited to, conductive hearing loss and sensorineural hearing loss.

The conductive hearing loss is a hearing loss caused by an ear disease, and is a hearing loss caused by problems in organs such as the eardrum and ossicles, which are organs that transmit sound.

The sensorineural hearing loss is a hearing loss caused by a problem in the cochlea, an organ that senses sound, the auditory nerve that transmits sound with electrical energy, and the brain responsible for hearing that plays comprehensive roles such as discrimination and understanding of sound, and the like. The cause of sensorineural hearing loss may be hearing loss caused by noise, drugs, aging, trauma, and the like, for example, ototoxic hearing loss. The ototoxic hearing loss may be caused by administration of any one or more ototoxic drugs selected from the group consisting of gentamicin, streptomycin, kanamycin, neomycin, amikacin, tobramycin, netilmicin, dibekacin, sisomycin, livodomycin, cisplatin, carboplatin, and oxaliplatin.

In addition, the hearing loss may include noise-induced hearing loss, age-related hearing loss, sudden hearing loss, auditory neuropathy due to diabetes, ototoxic hearing loss, traumatic hearing loss, viral hearing loss, and the like.

However, if hearing is impaired or lost, it is not limited to the scope of the above-described hearing loss of the present disclosure but may be included in the scope of diseases.

In the present disclosure, “tinnitus” refers to perception of sound in the absence of an external source of an acoustic signal, and may include, but is not limited to, objective tinnitus, subjective tinnitus, peripheral tinnitus, and central tinnitus. Specifically, tinnitus may include, but is not limited to, subjective tinnitus caused by various causes such as noise, drugs, aging, trauma, viruses, and the like.

The objective tinnitus (or non-subjective tinnitus) is tinnitus that is audible even from the outside and subjective tinnitus (or conscious tinnitus) that is audible to the patient himself but is not audible from the outside. In addition, tinnitus may be classified into peripheral tinnitus and central tinnitus based on differences in how they are perceived by the affected individual. The peripheral (or cochlear) tinnitus is presumed to originate from the peripheral nervous system and cochlea, and the central tinnitus is presumed to originate from the auditory cortex.

The subjective tinnitus may have several causes, but it is usually caused by an otologic disorder that results in hearing loss. The most common cause is noise, which comes from exposure to excessive or loud noise. Further, tinnitus may be accompanied by a sudden hearing loss in the absence of obvious external factors. The subjective tinnitus is also known to be caused by side effects of some medicaments, such as aspirin. In addition, the subjective tinnitus may be caused by side effects of natural hearing impairment such as aging or hereditary (congenital) hearing impairment. Accordingly, tinnitus includes subjective tinnitus occurring by various causes such as noise, drugs, aging, trauma, viruses, and the like.

Tinnitus according to the present disclosure may be subjective tinnitus.

In the present disclosure, “extract” means a product such as a solid obtained by removing a solvent from a liquid component, the liquid component being obtained by immersing a target substance in various solvents and then extracting the substance for a predetermined period of time at room temperature or in a warm state. Further, in addition to the product, the extract may include the extract itself and all types of substances capable of being prepared using the extract such as a dilution of the product, a concentrate thereof, a prepared product thereof, a purified product, and the like.

An extraction method for preparing the extract is not particularly limited, and may be performed according to a method commonly used in the art. An extraction temperature may be adopted by those skilled in the art in various temperature ranges suitable for the extraction method, but not limited thereto. In addition, the extraction time varies depending on the extraction method, an appropriate extraction time may be adopted by those skilled in the art, and the extraction may be performed in a single time or multiple times.

The extract obtained by extraction with the primary extraction solvent is obtained in a liquid form from which impurities are removed by filtration according to a conventional method, or in powder form by concentration under reduced pressure and/or drying the obtained extract in liquid form according to a conventional method. Further, the extraction process may further comprise, if necessary, obtaining a fraction having a high content of the active ingredient.

The sweet potato stem and/or leaf extract may be prepared by extraction from sweet potato stems or leaves according to a conventional method for preparing an extract. For example, the extraction may be performed with the primary extraction solvent, preferably water, C₁ to C₄ lower alcohol (for example, methanol, ethanol, propanol, isopropanol, butanol, or the like) or a mixed solvent thereof in an amount of about 1 to 25 times, preferably about 3 to 20 times the weight of the sweet potato stems or leaves.

The extraction may be performed by an extraction method known in the art, for example, cold extraction, hot water extraction, ultrasonic extraction, reflux cooling extraction, or the like, but is not limited thereto.

The extraction temperature may be adopted by those skilled in the art in various temperature ranges suitable for the extraction method, and for example, may be 20° C. to 100° C., and the like, but is not limited thereto.

In addition, the extraction time varies depending on the extraction method, and an appropriate extraction time may be adopted by those skilled in the art. The extraction may be performed single or multiple times in the range of about 1 hour to 10 days, but is not limited thereto.

The extract obtained by extraction with the primary extraction solvent may be obtained in a liquid form from which impurities are removed by filtration according to a conventional method, or in powder form by concentration under reduced pressure and/or drying the obtained extract in liquid form according to a conventional method.

Before the extraction process, the extraction may be carried out after performing an enzyme treatment with a conventional cell wall degrading enzyme for removing the cell wall, and the like, ultrasonic treatment, vortexing treatment, and the like.

Further, the extraction process may further comprise, if necessary, obtaining a fraction having a high content of the active ingredient. In other words, after dispersing the extract obtained by extraction with the primary extraction solvent in water, extraction with an appropriate secondary extraction solvent, for example, water-saturated C₁-C₄ alcohol, may be performed to increase the content of the active ingredient.

When the sweet potato stem extract and the sweet potato leaf extract are comprised together, the extract may be an extract obtained by extracting stems and leaves of sweet potatoes separately and combining the resulting extract, or an extract obtained by extracting stem and leaves of sweet potatoes together.

Further, the extraction process may further comprise, if necessary, obtaining a fraction having a high content of the active ingredient. In other words, after dispersing the extract obtained by extraction with the primary extraction solvent in water, extraction with an appropriate secondary extraction solvent, for example, saturated C₁-C₄ alcohol, may be performed to increase the content of the active ingredient.

In the present disclosure, “fraction” refers to a result obtained by performing fractionation in order to separate a specific component or a specific group of components from a mixture including various components.

A fractionation method for preparing the fraction is not particularly limited, and may be performed according to a method commonly used in the art. For example, the fractionation method may be a solvent fractionation method performed by treating various solvents, an ultrafiltration fractionation method performed by passing through an ultrafiltration membrane having a constant molecular weight cut-off value, a chromatographic fractionation method in which various chromatographies (those designed for separation depending on size, charge, hydrophobicity or affinity) are performed, and a combination thereof, and the like. The kind of solvent used to obtain the fraction in the present disclosure is not particularly limited, and may be any solvent known in the art. Non-limiting examples of the fractionation solvent may include water, an organic solvent, or a mixed solvent thereof. The organic solvent may be an alcohol having 1 to 4 carbon atoms, a polar solvent such as ethyl acetate or acetone, a non-polar solvent such as hexane or dichloromethane, or a mixed solvent thereof.

According to an embodiment of the present disclosure, the sweet potato stem or leaf extract was prepared by extraction with ethanol. More preferably, the sweet potato stem or leaf extract was prepared by extraction with an aqueous ethanol solution.

Specifically, the sweet potato stem or leaf extract was extracted with about 50 to 90% of an aqueous ethanol solution.

More specifically, the extract may be obtained by extraction twice at 80 to 100° C. with about 60 to 80% aqueous ethanol solution, preferably about 70% aqueous ethanol solution, followed by concentration.

It is preferable to include the sweet potato stem and/or leaf extract in an amount of 0.1 to 95% by weight based on the total weight of the composition containing the extract of sweet potato stems, leaves, or both of the present disclosure as an active ingredient, but is not limited thereto.

The term “prevention” as used herein refers to any action that suppresses tinnitus or delays the onset of tinnitus by administration of the pharmaceutical composition according to the present disclosure.

The term “treatment” as used herein refers to any action in which symptoms of tinnitus are improved or beneficially changed by administration of the pharmaceutical composition according to the present disclosure.

The pharmaceutical composition of the present disclosure may comprise a pharmaceutically acceptable carrier, and may be formulated as oral dosage forms such as powders, granules, tablets, capsules, suspensions, emulsions, syrups and aerosols, and the like, and forms of external preparations, suppositories, and sterile injection solutions each according to the general method.

Examples of the pharmaceutically acceptable carrier may include, but are not limited to, those commonly used in the art such as lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil, and the like. In addition, the pharmaceutical composition of the present disclosure may include, but is not limited to, diluents or excipients such as fillers, extenders, binders, wetting agents, disintegrants, surfactants, and other pharmaceutically acceptable additives.

When the pharmaceutical composition of the present disclosure is formulated as a solid preparation for oral use, the pharmaceutical composition includes tablets, pills, powders, granules, capsules, and the like. These solid preparations may include, but is not limited to, at least one or more excipients, for example, starch, calcium carbonate, sucrose or lactose, gelatin, and the like, lubricants such as magnesium stearate, talc, and the like.

When the pharmaceutical composition of the present disclosure is formulated as a liquid formulation for oral use, the composition includes, but is not limited to, suspensions, internal solutions, emulsions, syrups, and the like, and includes diluents such as water, liquid paraffin, and the like, wetting agents, sweeteners, fragrances, and preservatives, and the like.

When the pharmaceutical composition of the present disclosure is formulated for parenteral use, the composition includes sterile aqueous solutions, non-aqueous solvents, suspensions, emulsions, freeze-dried preparations, and suppositories, wherein the non-aqueous solvent and suspension include, but are not limited to, propylene glycol, polyethylene glycol, vegetable oils such as olive oils, injectable esters such as ethyl oleate, and the like. As the suppository base, witepsol, macrogol, tween 61, cacao butter, laurin, glycerogelatin, and the like, may be used, but the present disclosure is not limited thereto.

The dose of the extract of sweet potato stems, leaves, or both contained in the pharmaceutical composition of the present disclosure varies depending on the patient's condition and weight, age, disease severity, drug form, administration route and period, but may be appropriately selected by those skilled in the art. For example, the extract of sweet potato stems, leaves, or both may be administered at a dose of 0.0001 to 100 mg/kg per day, preferably 0.001 to 10 mg/kg, wherein the administration may be performed once or several times a day.

The pharmaceutical composition of the present disclosure may be administered to mammals such as rats, mice, livestock, humans, and fish such as zebrafish, by various routes, for example, oral, intraperitoneal or intravenous, intramuscular, subcutaneous, intrauterine or intracerebrovascular injection.

The pharmaceutical composition of the present disclosure may contain 0.01 to 95% by weight, preferably 1 to 80% by weight of the extract of sweet potato stems, leaves, or both, based on the total weight of the composition. In addition, the extract of sweet potato stems, leaves, or both contained in the pharmaceutical composition of the present disclosure may be obtained by the same or similar method as the above-described extraction method or fractionation method, but is not limited thereto.

The pharmaceutical composition for preventing or treating an ear disease of the present disclosure has an excellent therapeutic effect on hearing loss by reducing abnormal behavior due to hearing loss. In particular, the extract of sweet potato stems, leaves, or both has a remarkable effect in preventing and treating hearing loss, and have high economic utility by employing a material that has not been used as medicinal or health functional food until now.

The pharmaceutical composition for preventing or treating an ear disease of the present disclosure has an excellent therapeutic effect on tinnitus by reducing abnormal behavior due to tinnitus. In particular, the extract of sweet potato stems, leaves, or both has a remarkable effect in preventing and treating tinnitus, and have high economic utility by employing a material that has not been used as medicinal or health functional food until now.

Another embodiment of the present disclosure provides a method for preventing or treating an ear disease, comprising administering the extract or the pharmaceutical composition of the sweet potato stems, leaves, or both to a subject.

The pharmaceutical composition and ear disease are the same as described above.

The term “individual” as used herein refers to all animals, except humans, that have or may develop an ear disease, and typically, may be an animal capable of exhibiting a beneficial effect by treatment with a pharmaceutical composition comprising an extract or a fraction of sweet potato stems, leaves, or both as an active ingredient, but includes, without limitation, any individual who has or is likely to have symptoms of ear diseases.

As described above, the ear disease may be effectively prevented or treated by administering the pharmaceutical composition of the present disclosure to an individual. The pharmaceutical composition of the present disclosure may be administered as an individual therapeutic agent, or may be administered in combination with a conventional therapeutic agent for an ear disease, and may be administered sequentially or simultaneously with the conventional therapeutic agent.

The term “administration” of the present disclosure means introducing a predetermined substance to a patient by an appropriate method, and the administration route of the composition may be formed through any general route as long as the composition is able to reach a target tissue. In addition, the pharmaceutical composition of the present disclosure may be administered by any device capable of transporting the active substance to a target tissue.

The pharmaceutical composition of the present disclosure may be administered as an individual therapeutic agent, or may be administered in combination with a conventional therapeutic agent, and may be administered sequentially or simultaneously with the conventional therapeutic agent. In consideration of all of the above factors, the composition may be administered in a minimal amount without side effects, but in an amount capable of obtaining the maximum effect, which may be readily determined by those skilled in the art.

The present disclosure also provides a food composition for preventing or treating an ear disease, comprising an extract of sweet potato stems, leaves, or both as an active ingredient.

The term “improvement” as used herein refers to any action in which the ear disease is improved or beneficially changed by administration of the composition of the present disclosure.

The extract of sweet potato stems, leaves, or both and the ear diseases are the same as described above.

The food composition of the present disclosure may comprise conventional food additives, and whether or not it is suitable as a “food additive” is judged according to specifications and standards for the relevant item in accordance with general rules and general test methods, and the like, of the Food Additives Codex approved by the Ministry of Food and Drug Safety, unless otherwise specified.

Items listed in the “Food Additives Codex” may include, for example, chemical compounds such as ketones, glycine, potassium citrate, nicotinic acid, cinnamic acid, and the like, natural additives such as persimmon color, licorice extract, crystalline cellulose, Kaoliang Color, guar gum, and the like, mixed preparations such as sodium L-glutamate preparation, noodle-added alkali agent, preservative agent, tar color agent, and the like.

Further, the food composition of the present disclosure may be prepared and processed in the form of tablets, capsules, powders, granules, liquids, pills, and the like, for the purpose of preventing and/or improving an ear disease.

The food composition of the present disclosure may be used as a health functional food. The term “health functional food” means a food manufactured and processed using raw materials or ingredients having useful functionality for the human body according to the Health Functional Food Act, and the term “functionality” means ingestion for the purpose of obtaining effects useful for health purposes such as nutrient control or physiological action on the structure and function of the human body.

For example, the health functional food in the form of tablets may be manufactured by granulating a mixture of the extract of sweet potato stems, leaves, or both, an excipient, a binder, a disintegrant, and other additives by a conventional method, followed by compression molding by adding a lubricant or the like or by direct compression molding of the mixture. In addition, the health functional food in the form of tablets may contain a flavoring agent, and the like, and may be coated with a suitable coating agent, if necessary.

Among health functional foods in the form of a capsule, a hard capsule formulation may be prepared by filling a conventional hard capsule with a mixture containing the extract of sweet potato stems, leaves, or both, and additives such as excipients, or granules thereof or coated granules, and a soft capsule formulation may be prepared by filling a capsule base such as gelatin with a mixture containing a mixture containing the extract of sweet potato stems, leaves, or both, and additives such as excipients. The soft capsule formulation may contain a plasticizer such as glycerin or sorbitol, a colorant, a preservative, and the like, if necessary.

A health functional food in the form of a pill may be prepared by molding the mixture of the extract of sweet potato stems, leaves or both, excipients, binders, disintegrants, and the like, according to an appropriate method, and, if necessary, may be coated with sucrose or other suitable coating agent, or may be powdered with starch, talc or any suitable material.

A health functional food in the form of a granule may be prepared in a granular form according to an appropriate method by employing a mixture of the extract of sweet potato stems, leaves, or both, excipients, binders, disintegrants, and the like, and if necessary, may contain a fragrance ingredient, a flavoring agent, and the like. In the health functional food in the form of granules when performing the following particle size test using No. 12 (1680 μm), No. 14 (1410 μm) and No. 45 (350 μm) sieves, the total amount of granules may pass through No. 12 sieve and the granules remaining after passing through No. 14 sieve may have an amount of 5.0% or less of the total amount, and the granules passing through No. 45 sieve may have an amount of 15.0% or less of the total amount.

Definitions for terms such as excipients, binders, disintegrants, lubricants, flavoring agents, fragrance ingredients, and the like are described in documents known in the art, and include those having the same or similar functions (Korean Pharmacopoeia Commentary, Moonseong publisher, Association of Korean Pharmacopoeia, 5th Revision, p33-48, 1989).

There is no particular limitation on the type of the food, and examples of foods to which the extract of sweet potato stems, leaves or both of the present disclosure is able to be added include beverages, gums, vitamin complexes, drinks, and the like, and include all health functional foods in a conventional sense.

The food composition of the present disclosure may contain 0.01 to 95% by weight, preferably 1 to 80% by weight of the extract of sweet potato stems, leaves, or both, based on the total weight of the composition. In addition, the extract of sweet potato stems, leaves, or both contained in the food composition of the present disclosure may be obtained by the same or similar method as the extraction method or fractionation method described in the preparation of the pharmaceutical composition, but is not limited thereto.

Still another embodiment of the present disclosure provides a health functional food comprising the food composition. The food composition and the health functional food are the same as described above.

The present disclosure also provides a composition for use in treatment of an ear disease, comprising an extract of sweet potato stems, leaves or both.

The present disclosure also provides use of an extract of sweet potato stems, leaves or both for preparation of a medicament for treatment of an ear disease.

The present disclosure also provides a method of treating an ear disease, comprising administering an extract of sweet potato stems, leaves or both to a subject having an ear disease.

The subject refers to an animal, and typically may be a mammal capable of exhibiting beneficial effects by treatment with the extract of sweet potato stems, leaves, or both of the present disclosure. Preferred examples of the subject may include primates such as humans. Further, the subject may include all subjects having an ear disease or at risk of having symptoms of the ear disease.

The present disclosure also provides a composition for use in prevention or treatment of an ear disease, comprising an extract of sweet potato stems, leaves or both. The present disclosure also provides use of an extract of sweet potato stems, leaves or both for preparation of a medicament for prevention or treatment of an ear disease.

In the present disclosure, terms such as “extract of sweet potato stems, leaves or both”, “ear disease” and “administration” are the same as described above.

### Advantageous Effects

The extract of sweet potato stems, leaves or both according to the present disclosure may suppress hearing loss by effectively inhibiting the increase in hearing threshold and the hair cell damage due to hearing loss, which is useful for prevention or treatment of hearing loss, and may be effectively used in pharmaceutical compositions or health functional foods for treatment or prevention of an ear disease such as hearing loss or tinnitus by inhibiting the behavior, and the like, caused by the tinnitus in a tinnitus-inducing animal model.

### DESCRIPTION OF DRAWINGS

FIG. 1 shows the prevention, treatment and improvement effects of a sweet potato stem extract (stems of Ipomoea batatas shown as IBS in the drawings) confirmed by treating hair cells damaged by neomycin with the sweet potato stem extract (NOR: neomycin untreated normal control group, NM: neomycin-induced untreated control group, and IBS 1 μg/mL: 1 μg/mL of sweet potato stem extract-treated group);

FIG. 2 shows the prevention, treatment and improvement effects of a sweet potato leaf extract (leaves of Ipomoea batatas shown as IBL in the drawings) confirmed by treating hair cells damaged by neomycin with the sweet potato leaf extract (NOR: neomycin untreated normal control group, NM: neomycin-induced untreated control group, and IBL 1 μg/mL: 1 μg/mL of sweet potato leaf extract-treated group);

FIG. 3 shows the comparison of prevention, treatment and improvement effects of a sweet potato tuberous root extract (tuberous roots of Ipomoea batatas shown as IB in the drawings), a sweet potato stem extract (IBS), a sweet potato leaf extract (IBL) obtained by treating hair cells damaged by neomycin with the sweet potato tuberous root extract, the sweet potato stem extract, and the sweet potato leaf extract (NOR: neomycin untreated normal control group, NM: neomycin-induced untreated control group, IB 1 μg/mL: 1 μg/mL of sweet potato tuberous root extract-treated group, IBS 1 μg/mL: 1 μg/mL of sweet potato stem extract-treated group, and IBL 1 μg/mL: 1 μg/mL of sweet potato leaf extract-treated group);

FIG. 4 shows the effect of the sweet potato stem extract on hearing threshold after exposure to noise confirmed by presenting a click stimulus sound during an auditory brainstem response test (NIHL, i.e., noise-induced hearing loss: untreated control group after exposure to noise, and IBS 300 mg/kg: experimental group treated with 300 mg/kg of sweet potato stem extract after exposure to noise);

FIG. 5 shows the effect of the sweet potato stem extract on hearing threshold after exposure to noise confirmed by presenting a 16 kHz pure tone of stimulus sound during the auditory brainstem response test (NIHL: untreated control group after exposure to noise, and IBS 300 mg/kg: experimental group treated with 300 mg/kg of sweet potato stem extract after exposure to noise);

FIG. 6 shows the effect of the sweet potato leaf extract on hearing threshold after exposure to noise confirmed by presenting a click stimulus sound during the auditory brainstem response test (NIHL: untreated control group after exposure to noise, IBL 300 mg/kg: experimental group treated with 300 mg/kg of sweet potato leaf extract after exposure to noise);

FIG. 7 shows the effect of the sweet potato leaf extract on hearing threshold after exposure to noise confirmed by presenting a 16 kHz pure tone of stimulus sound during the auditory brainstem response test (NIHL: untreated control group after exposure to noise, and IBL 300 mg/kg: experimental group treated with 300 mg/kg of sweet potato leaf extract after exposure to noise)

FIG. 8 shows the time taken to pass through a transparent tube underwater and the number of rotations(turning) in the transparent tube when evaluating the sweet potato stem extract (stems of Ipomoea batatas) based on the behavior in an animal model of tinnitus (NOR: tinnitus non-induced control group, SS: tinnitus alone-induced group, IBS 10: experimental group treated with 10 μg/ml of sweet potato stem extract);

FIG. 9 shows SA ratio results of the tinnitus alone-induced group and the experimental group treated with the sweet potato stem extract in the tinnitus-induced group when evaluating the effect of the sweet potato stem extract in the animal model of tinnitus (SS: tinnitus alone-induced group, and SS+IBS: experimental group treated with sweet potato stem extract in tinnitus-induced group); and

FIG. 10 is a graph illustrating results of measuring the hearing amplitude using the auditory brainstem response with the click stimulus sound after exposure to noise (NOR: tinnitus non-induced control group, SS: tinnitus alone-induced group, and SS+IBS: experimental group treated with sweet potato stem extract in tinnitus-induced group).

### BEST MODE

Hereinafter, the present disclosure will be described in more detail through Examples. However, these Examples and Experimental Examples are provided for illustrating the present disclosure, and the scope of the present disclosure is not limited to these Examples and Experimental Examples.

### Example 1: Preparation of Sweet Potato Stem Extract (Stems of Ipomoea batatas)

500 g of sweet potato stems were subjected to primary extraction with 9 L of 70% ethanol at 90° C. for 120 minutes. 4.5 L of 70% ethanol was added again to the primary extract, and the resulting product was secondly extracted at the same temperature for 60 minutes. Then, the extract was filtered and concentrated at 70-80 rpm in a water bath at 40° C. The concentrate was frozen at −50° C. and then freeze-dried for 7 days. After freeze-drying, the product was stored in −50° C. freezer. As a result of the extraction of the total of 500 g of sweet potato stems, it was confirmed that 7.27 g of the extract was obtained, and the yield was 1.5%.

### Example 2: Preparation of Sweet Potato Leaf Extract (Leaves of Ipomoea batatas)

500 g of sweet potato leaves were subjected to primary extraction with 9 L of 70% ethanol at 90° C. for 120 minutes. 4.5 L of 70% ethanol was added again to the primary extract, and the resulting product was secondly extracted at the same temperature for 60 minutes. Then, the extract was filtered and concentrated at 70-80 rpm in a water bath at 40° C. The concentrate was frozen at −50° C. and then freeze-dried for 7 days. After freeze-drying, the product was stored in −50° C. freezer. As a result of the extraction of the total of 500 g of sweet potato leaves, it was confirmed that 14 g of the extract was obtained, and the yield was 2.8%.

### Comparative Example 1: Preparation of Sweet Potato Tuberous Root Extract (Tuberous Roots of Ipomoea batatas)

500 g of sweet potato tuberous roots were primarily extracted with 9 L of 70% ethanol at 90° C. for 120 minutes. 4.5 L of 70% ethanol was added again to the primary extract, and the resulting product was secondly extracted at the same temperature for 60 minutes. Then, the extract was filtered and concentrated at 70-80 rpm in a water bath at 40° C. The concentrate was frozen at −50° C. and then freeze-dried for 7 days. After freeze-drying, the product was stored in −50° C. freezer. As a result of the extraction of the total of 500 g of sweet potato tuberous roots, it was confirmed that 9 g of the extract was obtained, and the yield was 1.8%.

Experimental Example 1: Protective effect of sweet potato stem extract (stems of Ipomoea batatas) on hair cells in ototoxic zebrafish model hair cells reduced by neomycin were treated with the sweet potato stem extract of Example 1 to confirm a protective effect thereof on the hair cells.

Specifically, zebrafish larvae on Day 6 post fertilization were placed in 24 wells and exposed to 2 μM neomycin for 1 hour. Thereafter, the exposed larvae were exposed with 1 μg/mL of the sweet potato stem extract of Example 1 for 6 hours, and a 0.03% sea salt solution was treated as a control group. For direct observation of hair cells, a fluorescence microscope (Olympus 1×70, Olympus, Japan) was used and the cells were stained with 0.1% YO-PRO for 30 minutes. The zebrafish larvae were anesthetized with 0.02% tricaine, and the hair cells were observed with a fluorescence microscope. Data were analyzed by counting the number of hair cells obtained through the fluorescence microscope.

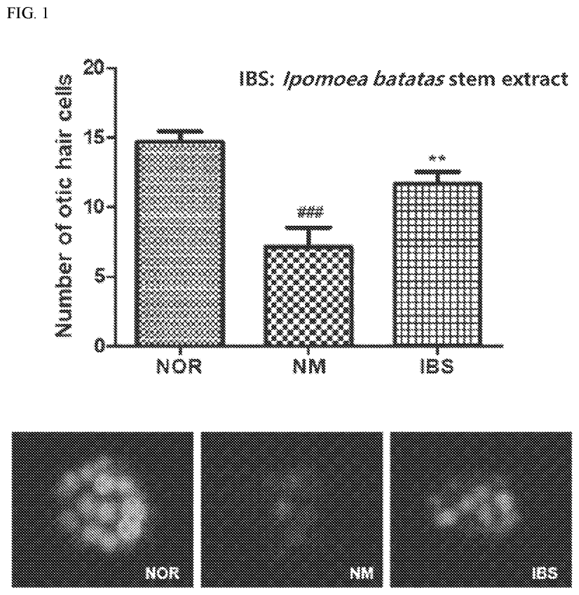

Results thereof are shown in FIG. 1.

As shown in FIG. 1, it was confirmed that the number of hair cells significantly increased in the experimental group treated with the sweet potato stem extract, and from this confirmation, it was proved that the sweet potato stem extract according to the present disclosure had an excellent effect in the prevention, improvement and treatment of hearing loss.

Experimental Example 2: Protective Effect of Sweet Potato Leaf Extract (Leaves of Ipomoea batatas) on Hair Cells in Ototoxic Zebrafish Model

hair cells reduced by neomycin were treated with the sweet potato leaf extract of Example 1 to confirm a protective effect thereof on the hair cells.

Specifically, zebrafish larvae on Day 6 post fertilization were placed in 24 wells and exposed to 2 μM neomycin for 1 hour. Thereafter, the exposed larvae were exposed with 1 μg/mL of the sweet potato leaf extract of Example 1 for 6 hours, and a 0.03% sea salt solution was treated as a control group. For direct observation of hair cells, a fluorescence microscope (Olympus 1×70, Olympus, Japan) was used and the cells were stained with 0.1% YO-PRO for 30 minutes. The zebrafish larvae were anesthetized with 0.02% tricaine, and the hair cells were observed with a fluorescence microscope. Data were analyzed by counting the number of hair cells obtained through the fluorescence microscope.

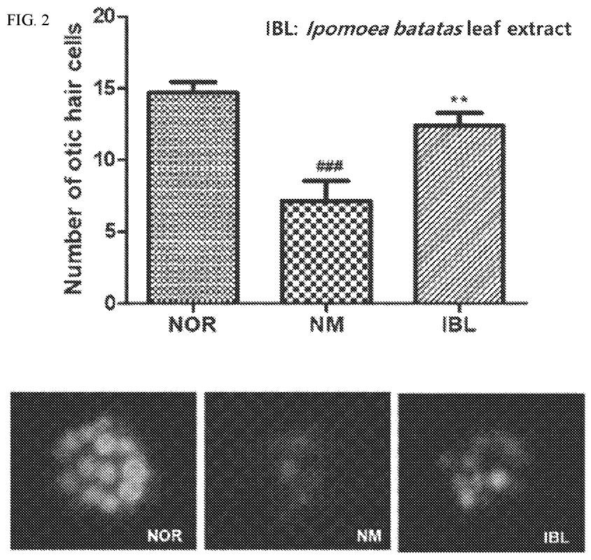

Results thereof are shown in FIG. 2.

As shown in FIG. 2, it was confirmed that the number of hair cells significantly increased in the experimental group treated with the sweet potato leaf extract, and from this confirmation, it was proved that the sweet potato leaf extract according to the present disclosure had an excellent effect in the prevention, improvement and treatment of hearing loss.

Experimental Example 3: Comparison of protective effect on hair cells damaged by neomycin among sweet potato tuberous root extract (tuberous roots of Ipomoea batatas), sweet potato stem extract, and sweet potato leaf extract in ototoxic zebrafish model

hair cells reduced by neomycin were treated with the sweet potato stem extract of Example 1, the sweet potato leaf extract of Example 2, and the sweet potato tuberous root extract of Comparative Example 1 to confirm protective effects thereof on the hair cells.

Specifically, zebrafish larvae on Day 6 post fertilization were placed in 24 wells and exposed to 2 μM neomycin for 1 hour. Thereafter, the exposed larvae were exposed with 1 μg/mL of the sweet potato stem extract of Example 1, the sweet potato leaf extract of Example 2, and the sweet potato tuberous root extract of Comparative Example 1, respectively, for 6 hours, and a 0.03% sea salt solution was treated as a control group. For direct observation of hair cells, a fluorescence microscope (Olympus 1×70, Olympus, Japan) was used and the cells were stained with 0.1% YO-PRO for 30 minutes. The zebrafish larvae were anesthetized with 0.02% tricaine, and the hair cells were observed with a fluorescence microscope. Data were analyzed by counting the number of hair cells obtained through the fluorescence microscope.

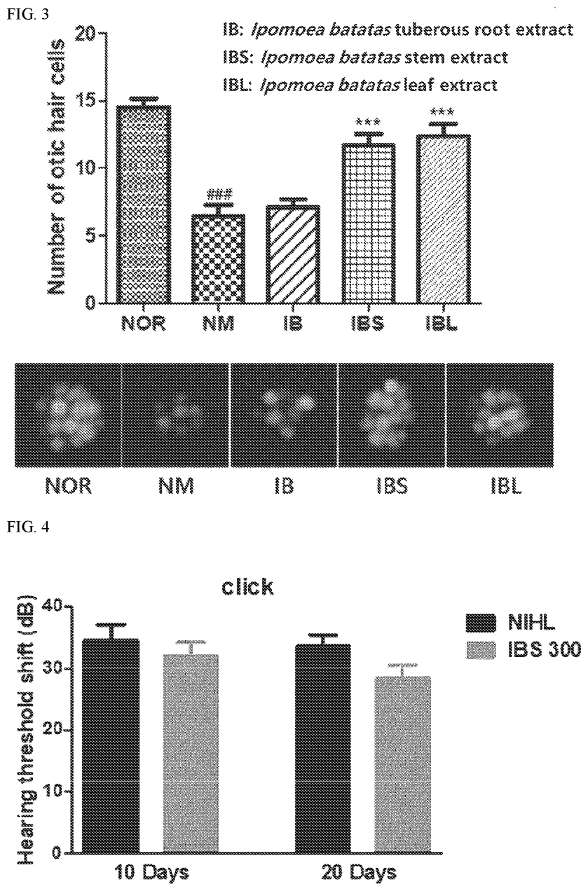

Results thereof are shown in FIG. 3.

As shown in FIG. 3, it was confirmed that the experimental group treated with the sweet potato tuberous root extract did not increase the number of hair cells, indicating that there was no effect on hearing loss. On the other hand, it was confirmed that the sweet potato stem and leaf extracts according to the present disclosure had an excellent effect in the prevention, improvement and treatment of hearing loss.

Experimental Example 4: Confirmation of Improvement in Hearing Loss after Exposure to Noise by Sweet Potato Stem Extract

Experimental Example 4-1. Confirmation of Hearing Threshold Using Click Broadband Click Stimulus Sound

In order to verify an effect of sweet potato stem extract on a hearing threshold after exposure to noise, an experiment for measuring the hearing threshold was performed using an auditory brainstem response. A method of measuring the auditory brainstem response (ABR) is a method of evaluating a response to a sound by measuring electric energy when a sound stimulus is transmitted as an electrical signal in the auditory nerve. When the sound reaches the auditory nerve through the outer ear, the middle ear, and the cochlea, the response reflects all states of the outer ear, the middle ear, and the cochlea, which reflects the actual sound energy to which the sound energy reaches the brain. The hearing threshold refers to the minimum sensory point of sound that is barely audible. In a normal mouse, the response is observed even to a sound as low as 20 dB on average.

Specifically, rats to which 300 mg/kg of sweet potato stem extract (IBS) prepared in Example 1 was administered and untreated control rats were divided into three groups of eight rats, respectively, and evaluated. The rats were exposed to a noise, specifically, a 115 dB complex sound, for 90 minutes, and after 24 hours from exposure to noise, the sweet potato stem extract of Example 1 was orally administered at the same time every day. The hearing threshold was evaluated before exposure to noise, on Days 1, 10, and 20 after exposure.

For the auditory brainstem response test, the rats were anesthetized by intramuscular injection of ketamine (4.57 mg/kg) and xylazine (0.43 mg/kg), and then evaluated while maintaining body temperature at 37±0.5° C. During the auditory brainstem response test, the rats were evaluated with a broadband click stimulus sound as a stimulus sound by lowering the sound gradually by 5 dB from 80 dB, and the smallest sound of the response was set as the threshold.

Results thereof are shown in FIG. 4.

As shown in FIG. 4, the group administered with 300 mg/kg of the sweet potato stem extract (IBS) showed a decrease in threshold on Day 20 after exposure to noise, which was 5 dB lower than that of the control group. Thus, the effect of improving hearing was verified.

Experimental Example 4-2. Confirmation of Hearing Threshold Using 16 kHz TB Stimulus Sound

In the auditory brainstem response test, the rats were evaluated with a 16 kHz pure tone as a stimulus sound by lowering the sound gradually by 5 dB from 80 dB.

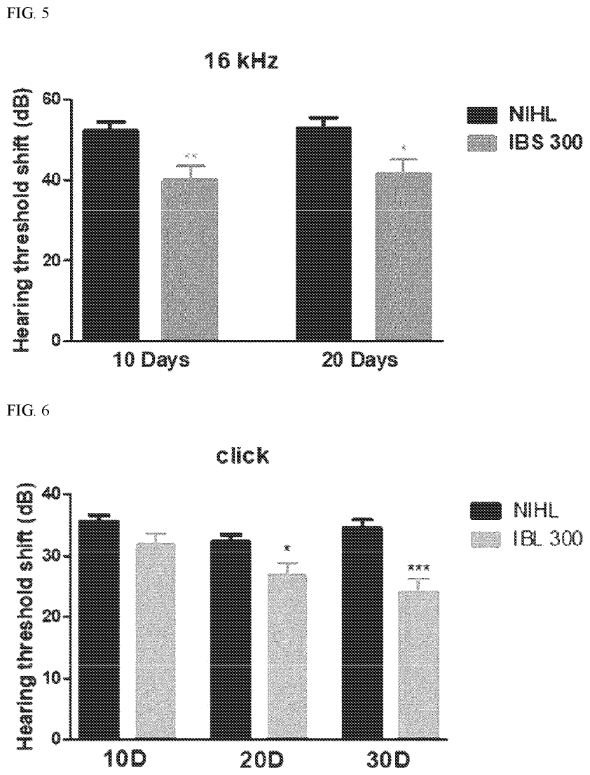

Results thereof are shown in FIG. 5.

As shown in FIG. 5, the group administered with 300 mg/kg of the sweet potato stem extract (IBS) showed a decrease in threshold on Day 10 and Day 20 after exposure to noise, which was 10 dB lower than that of the control group. Thus, the significant effect of improving hearing was verified.

Experimental Example 5: Confirmation of Improvement in Hearing Loss after Exposure to Noise by Sweet Potato Leaf Extract

Experimental Example 5-1. Confirmation of Hearing Threshold Using Broadband Click Stimulus Sound

In order to verify an effect of sweet potato leaf extract on a hearing threshold after exposure to noise, an experiment for measuring the hearing threshold was performed using an auditory brainstem response. A method of measuring the auditory brainstem response (ABR) is a method of evaluating a response to a sound by measuring electric energy when a sound stimulus is transmitted as an electrical signal in the auditory nerve. When the sound reaches the auditory nerve through the outer ear, the middle ear, and the cochlea, the response reflects all states of the outer ear, the middle ear, and the cochlea, which reflects the actual sound energy to which the sound energy reaches the brain. The hearing threshold refers to the minimum sensory point of sound that is barely audible. In a normal mouse, the response is observed even to a sound as low as 20 dB on average.

Specifically, rats to which 300 mg/kg of sweet potato leaf extract (IBL) prepared in Example 2 was administered and untreated control rats were divided into three groups of eight rats, respectively, and evaluated. The rats were exposed to a noise, specifically, a 115 dB complex sound, for 90 minutes, and after 24 hours from exposure to noise, the sweet potato leaf extract of Example 2 was orally administered at the same time every day. The hearing threshold was evaluated before exposure to noise, and on Days 1, 10, 20 and 30 after exposure.

For the auditory brainstem response test, the rats were anesthetized by intramuscular injection of ketamine (4.57 mg/kg) and xylazine (0.43 mg/kg), and then evaluated while maintaining body temperature at 37±0.5° C. During the auditory brainstem response test, the rats were evaluated by lowering the sound gradually by 5 dB from 80 dB with a broadband click stimulus sound, and the smallest sound of the response was set as the threshold.

Results thereof are shown in FIG. 6.

As shown in FIG. 6, the group administered with 300 mg/kg of the sweet potato leaf extract (IBL) showed a decrease in threshold on Day 20 and Day 30 after exposure to noise, which was 10 dB lower than that of the control group. Thus, the effect of improving hearing was verified.

Experimental Example 5-2. Confirmation of Hearing Threshold Using 16 kHz TB Stimulus Sound

In the auditory brainstem response test, the rats were evaluated with a 16 kHz pure tone as a stimulus sound by lowering the sound gradually by 5 dB from 80 dB.

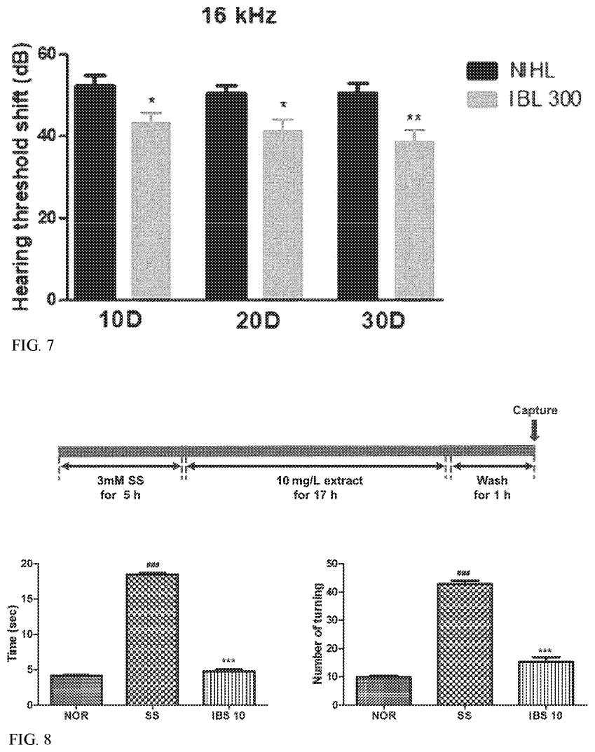

Results thereof are shown in FIG. 7.

As shown in FIG. 7, the group administered with 300 mg/kg of the sweet potato leaf extract (IBL) showed a decrease in threshold on Day 10, Day 20, and Day 30 after exposure to noise, which was 12 dB lower than that of the control group. Thus, the significant effect of improving hearing was verified.

Experimental Example 6: Tinnitus Inhibitory Effect of Sweet Potato Stem Extract (Stems of Ipomoea batatas) in Zebrafish Model Suffering from Salicylic Acid-Induced Tinnitus

The treatment effect on tinnitus was confirmed by employing a behavioral response test that is able to induce and evaluate tinnitus in zebrafish without physical stress.

Specifically, the zebrafish larvae were divided into a tinnitus non-induced control group (NOR), an experimental group in which tinnitus was induced by exposure to 3 mM salicylic acid for 5 hours (SS), and an experimental group exposed to the sweet potato stem extract prepared in Example 1 for 17 hours immediately after exposure to 3 mM salicylic acid for 5 hours (IBS). Here, for the IBS experimental group, the experiment was performed by exposing the zebrafish larvae to a concentration of 10 μg/ml of the sweet potato stem extract prepared in Example 1 (IBS 10). All experiments were carried out using a 0.03% sea salt solution as a base solution, and all experimental groups were acclimatized to 0.03% sea salt solution for 1 hour immediately before measurement.

In order to confirm the effect of the sweet potato stem extract prepared in Example 1, the zebrafish exposed to each solution and sweet potato stem extract were allowed to pass through a transparent tube (outer diameter of 25 mm, inner diameter of 21 mm, and total length of 60 cm) located in a water bath (75(W)×45(D)×45(H) cm), and the time taken to pass through the transparent tube and the number of rotations in the transparent tube were analyzed. The water temperature was maintained at 28° C. by installing an underwater heater in the water bath.

Results thereof are shown in FIG. 8.

As shown in FIG. 8, it could be confirmed that in the SS group as compared to the NOR group, tinnitus was induced as the time taken to pass through the transparent tube and the number of rotations in the transparent tube increased significantly (###p<0.001 vs. NOR). In the comparison on IBS efficacy, it was confirmed in the IBS 10 group that tinnitus was suppressed as the time taken to pass through the transparent tube and the number of rotations in the transparent tube were significantly lowered (***p<0.001 vs. SS).

Experimental Example 7: Skinner behavioral analysis of sweet potato stem extract (stems of Ipomoea batatas) in rat model suffering from salicylic acid-induced tinnitus

In the Skinner behavioral analysis with trained rats, tinnitus-induced animals tend to have an increased trigger-pressing response in the absence of cue tone. At this time, tinnitus induction may be confirmed in animals by mistaking the tinnitus sound as the cue tone and pressing the trigger. Here, a true positive refers to the number of times the trigger is pressed to acquire food when the cue tone is provided, and a false positive refers to the number of times the trigger is pressed when the cue tone is not provided.

Rats were divided into three groups each having three rats: a tinnitus non-induced control group (NOR), a tinnitus-induced group (SS), and an experimental group administered with sweet potato stem extract (SS+IBS), and evaluated with the sweet potato stem extract prepared in Example 1.

In the experiment, the SS group and the IBS group used salicylic acid, which is generally used for a tinnitus-inducing animal model, wherein the salicylic acid was orally administered at a dose of 350 mg/kg 3 hours before the test. The SS group was tested by oral administration of only salicylic acid, and the SS+IBS group was orally administered with salicylic acid 1 hour and 30 minutes before the test with 100 mg/kg of the sweet potato stem extract prepared in Example 1 after inducing tinnitus.

The test schedule was carried out as follows: 1 day before administration (Base), 3 days (Days 1-3) after simultaneous administration with salicylic acid, and 3 days (Days 4-6) during the tinnitus recovery period. For the SS group, on days 1 to 3, salicylic acid was administered 3 hours before the test and then water was administered 1 hour and 30 minutes before the test, and on days 4 to 6, only water was orally administered 1 hour and 30 minutes before the test. For the SS+IBS group, on days 1 to 3, salicylic acid was administered 3 hours before the test, and the sweet potato stem extract was administered 1 hour and 30 minutes before the test, and on days 4 to 6, only the sweet potato stem extract was administered 1 hour and 30 minutes before the test.

For behavioral response analysis, silence activity ratio (SA ratio) and false positive ratio (FP ratio) values were used. The SA ratio is calculated by dividing the number of responses when there is no cue tone by the number of responses during the time the cue tone is provided, and the FP ratio is calculated as the ratio of the number of responses in the absence of cue tone to the total number of responses.

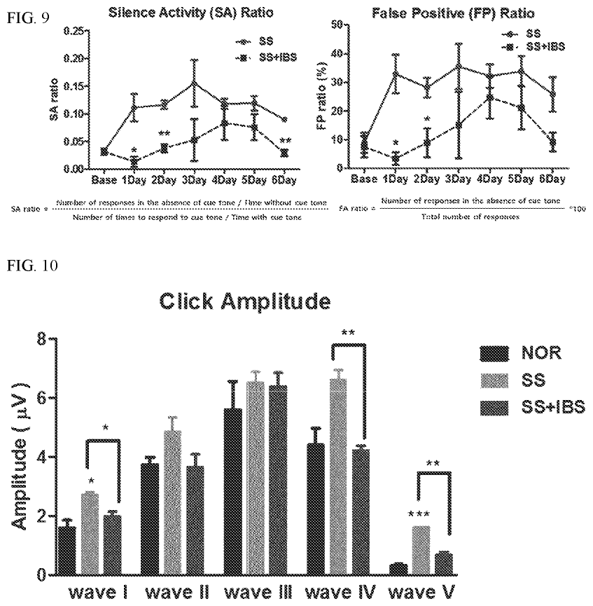

Results thereof are shown in FIG. 9.

As shown in FIG. 9, it could be confirmed in the SS group that tinnitus was induced by significantly increasing the SA ratio and FP ratio compared to the Base (Day 0). In the SS+IBS group administered orally with the sweet potato stem extract, the SA ratio and the FP ratio were significantly decreased compared to SS group on Day 1 and 2, confirming that tinnitus was suppressed (*p<0.05 vs SS, **p<0.01 vs SS), and on Days 3 to 5, there was no significant decrease, but tinnitus symptoms tended to decrease compared to the SS group. In addition, it could be confirmed that the SA ratio was significantly decreased on Day 6, and the tinnitus symptoms were continuously suppressed when IBS was administered orally (**p<0.01 vs SS).

Experimental Example 8: Confirmation of Hearing Amplitude of Sweet Potato Stem Extract (Stems of Ipomoea batatas) in Tinnitus Rat Model

A hearing amplitude measurement experiment using an auditory brainstem response was performed to confirm the effect of sweet potato stem extract on the sense as to the presence or absence of the sound by measuring the hearing amplitude after inducing tinnitus.

A method of measuring the auditory brainstem response (ABR) is a method of evaluating a response to a sound by measuring electric energy when a sound stimulus is transmitted as an electrical signal in the auditory nerve. When the sound reaches the auditory nerve through the outer ear, the middle ear, and the cochlea, the response reflects all states of the outer ear, the middle ear, and the cochlea and reflects the actual sound energy to which the sound energy reaches the brain. The hearing threshold refers to the minimum sensory point of sound that is barely audible. In a normal mouse, the response is observed even to a sound as low as 20 dB on average.

When measuring hearing amplitude, 5 amplitudes are usually observed (unit: μV, Wave I to Wave V). Amplitude 1 to 3 (Wave I to III) are formed by auditory branches extending from the 8th cranial nerve and lower nerves. Here, amplitude 1 is generated from the dendrites of the auditory nerve fibers, amplitude 2 is generated from the cochlear nucleus, and amplitude 3 represents the activity level of the superior olivary complex that receives auditory information from the cochlear nucleus. In addition, amplitude 4 and amplitude 5 (Wave IV and V) are formed from the upper brainstem and are related to the lateral lemniscus.

The schedule and experimental group before the click stimulus test were the same as those of Example 7, and the click stimulus test was performed and evaluated on the 5th day after oral administration of the sweet potato stem extract.

For the auditory brainstem response test, the rats were anesthetized by intramuscular injection of ketamine (11.43 mg/kg) and xylazine (1.08 mg/kg), and then evaluated while maintaining body temperature at 37±0.5° C. During the auditory brainstem response test, the rats were evaluated by lowering the sound gradually from 90 dB by 5 dB with a broadband click stimulus sound, and the smallest sound of the response was set as the threshold.

Results thereof are shown in FIG. 10.

As shown in FIG. 10, the SS group showed an overall increase in amplitude compared to the NOR group, and in particular, it could be confirmed that tinnitus was induced by a significant increase in amplitudes 1 and 5 (*p<0.05 vs NOR, ***p<0.001 vs NOR). The SS+IBS group showed an overall decrease in amplitude compared to the SS group, and in particular, it could be confirmed that the tinnitus symptoms were suppressed by a significant decrease in amplitudes 1, 4, and 5 (*p<0.05 vs SS, **p<0.01 vs SS).

As described above, it could be confirmed that in the sweet potato stem extract group, the false positive ratio due to tinnitus during the behavioral response test was almost similar to that of normal group, and thus that tinnitus was suppressed. Therefore, the sweet potato stem extract is usable for the prevention, improvement and treatment of tinnitus.

[Preparation Example]

The following formulations were prepared using the sweet potato stem and/or leaf extract of Example 1. However, the following Preparation Examples are provided to illustrate the present disclosure, and the content of the present disclosure is not limited thereto.

Preparation Example 1: Preparation of Tablet

| | |
|---|---|
| Sweet potato stem and/or leaf extract | 1000 mg |
| Lactose | 100 mg |
| Starch | 100 mg |
| Magnesium stearate | appropriate amount |

A tablet was prepared by mixing and tableting the above-described ingredients according to a conventional tablet preparation method.

Preparation Example 2: Preparation of Liquid

| | | |
|---|---|---|
| Sweet potato stem and/or leaf extract | 1000 | mg |
| CMC-Na | 20 | g |
| Isomerized glucose | 20 | g |

| | |
|---|---|
| Lemon flavor | appropriate amount |

Purified water was added to adjust the total volume to 1000 ml. A liquid was prepared by mixing the above-described ingredients and filling the mixture in a brown bottle, followed by sterilization according to a conventional liquid preparation method.

Preparation Example 3: Preparation of Capsule

| | | |
|---|---|---|
| Sweet potato stem and/or leaf extract | 1000 | mg |
| Crystalline cellulose | 3 | mg |
| Lactose | 14.8 | mg |
| Magnesium stearate | 0.2 | mg |

A capsule was prepared by mixing the above-described ingredients and filling the mixture in a gelatin capsule according to a conventional capsule preparation method.

Preparation Example 4: Preparation of Injection

| | | |
|---|---|---|
| Sweet potato stem and/or leaf extract | 1000 | mg |
| Mannitol | 180 | mg |
| Sterile distilled water for injection | 2974 | mg |
| Na₂HPO₄12H₂O | 26 | mg |

An injection was prepared with the above component content per 1 ampoule (2 ml) according to a conventional injection preparation method.

Preparation Example 5: Preparation of Health Functional Beverage

| | | |
|---|---|---|
| Sweet potato stem and/or leaf extract | 100 | mg |
| Citric acid | 1000 | mg |
| Oligosaccharide | 100 | g |
| Plum Concentrate | 2 | g |
| Total content with purified water added | 900 | mL |

A health functional beverage was prepared by mixing the above-described ingredients according to a conventional health functional beverage preparation method.

1-18. (canceled)

19. A method of treating an ear disease, comprising administering a sweet potato stem extract and/or a sweet potato leaf extract to a subject having an ear disease.

20. The method of claim 19, wherein the sweet potato stem extract is Ipomoea batatas stem extract.

21. The method of claim 19, wherein the sweet potato leaf extract is Ipomoea batatas leaf extract.

22. The method of claim 19, wherein the ear disease is one selected from the group consist of hearing loss, tinnitus and deafness.

23. The method of claim 22, wherein the hearing loss is conductive hearing loss or sensorineural hearing loss.

24. The method of claim 22, wherein the hearing loss is one selected from the group consisting of noise-induced hearing loss, age-related hearing loss(presbycusis), sudden hearing loss, auditory neuropathy due to diabetes, ototoxic hearing loss, traumatic hearing loss, and viral hearing loss.

25. The method of claim 24, wherein the ototoxic hearing loss is caused by administration of any one or more ototoxic drugs selected from the group consisting of gentamicin, streptomycin, kanamycin, neomycin, amikacin, tobramycin, netilmicin, dibekacin, sisomycin, livodomycin, cisplatin, carboplatin, and oxaliplatin.

26. The method of claim 22, wherein the tinnitus is one selected from the group consisting of objective tinnitus, subjective tinnitus, peripheral tinnitus, and central tinnitus.

27. The method of claim 19, wherein the sweet potato stem extract and/or a sweet potato leaf extract is an extract of water, a C₁ to C₄ lower alcohol, or a mixed solvent thereof, or a fraction thereof.

28. The method of claim 27, wherein the C₁ to C₄ lower alcohol is one selected from the group consist of methanol, ethanol, propanol, isopropanol and butanol.

29. The method of claim 27, wherein the extract is an ethanol aqueous solution extract.

30. The method of claim 28, wherein the extract is an 50-90% of ethanol aqueous solution extract.
